(19) United States
(12) Patent Application Publication
(10) Pub. No.: US 2017/0056316 A1
(43) Pub. Date: Mar. 2, 2017

(54) COMPOSITIONS AND METHODS FOR DISGUISING DERMATOLOGICAL BLEMISHES

(71) Applicant: Silicone Arts Laboratories, Inc., Memphis, TN (US)

(72) Inventors: Oscar Polk Atkinson, Memphis, TN (US); Kevin Yacoub, Los Olivos, CA (US)

(21) Appl. No.: 14/834,494

(22) Filed: Aug. 25, 2015

(51) Int. Cl. A61K 8/89; A45D 40/26; A61Q 1/00; A61Q 1/02; A61K 8/58

(52) U.S. Cl. CPC A61K 8/89; A61Q 1/02; A61K 8/585; A61Q 1/00; A45D 40/26; A61K 2800/95; A61K 2800/43; A61K 2800/87; A61K 2800/592; A61K 2800/88

(57) ABSTRACT

Novel compositions for the disguising of facial blemishes and scars in human users are disclosed. In addition, methods for utilizing said compositions, as well as kits for packaging and holding products containing said compositions, are disclosed. The novel compositions disclosed are based on a silicone material which is pre-mixed with a catalyst and an adhesion promoter, and sometimes also with a flocking agent. These pre-mixed compositions represent a significant improvement in product simplicity and ease of use for the person using the product. The methods disclosed are for utilizing products made from the claimed compositions and include simple dispensing of the silicone mixture followed by application of a skin texturing pad. The prescribed use of the skin texturing pad in the claimed methods is essential to create texture. Finally, kits for dispensing the primer-less silicone mixtures and for packaging the products are disclosed.

### BACKGROUND OF THE INVENTION

This section is intended to introduce various aspects of the art, which may be associated with exemplary embodiments of the present disclosure. This discussion is believed to assist in providing a framework to facilitate a better understanding of particular aspects of the present disclosure. Accordingly, it should be understood that this section should be read in this light, and not necessarily as admissions of prior art.

Field of the Invention

The present invention relates to cosmetic systems. More specifically, the invention relates to a cosmetic system that may be used to obscure dermatological defects. The invention also relates to methods for disguising dermatological blemishes.

Discussion of Technology

Some persons carry a skin defect that may cause embarrassment. The defect may be the result of a surgical procedure such as a tracheostomy, leaving permanent scarring in the neck or a scar from a cosmetic procedure or scars produced by skin cancer removal. Alternatively, the defect may be a result of trauma such as an automobile accident, a fall, or an assault. Alternatively, the defect may be significant skin blemishes due to acne scarring. In any of these instances, the person may be self-conscious about their appearance and seek to disguise or camouflage the skin defect.

Cosmetic materials are well-known as a means of enhancing or otherwise altering a person's appearance. Cosmetics are used to cover inconsistent skin pigmentation and wrinkles. However, standard cosmetics are not always suitable where the skin defect is profound.

For significant physiological defects, prostheses such as maxillofacial prosthetics are known. Such prosthetics may be of particular use for patients who have undergone the removal of a highly visible portion of their anatomy such as an ear, the nose, or an ocular orbit due to cancer. Maxillofacial prosthetics may be too expensive or too cumbersome to be used in the case of “mere” skin defects.

### SUMMARY

The present invention provides compositions for concealing dermatological blemishes on a human user, as well as methods for disguising dermatological blemishes on a human user. The methods employ a cosmetic system in the form of a kit that is provided or sold to a user as a box or other package.

The composition includes a silicone material mixed with an adhesion promoter and a catalyst (hereafter referred to as “silicone mixture”). In one aspect, the silicone mixture contains a flocking agent. Flocking agents may be, but are not restricted to, a thixotropic agent or a thickener or both. In some embodiments variations of different pigments and fibers are used to match the skin tone and/or texture of the user. In one embodiment the tones are selected to match the skin of the user.

The method first includes opening the package. In one aspect, the package includes a single container. Residing within the first silicone container is a mixture of a viscous silicone material, adhesion promoter, and catalyst. In some embodiments the package contains two containers wherein a first container contains a viscous silicone material and an adhesion promoter and the second container contains a catalyst.

The package also includes at least one skin texturing pad. The skin texturing pad is fabricated with dimples for creating texture to the translucent elastic layer while the silicone material is curing. In this way, dimples are formed in the elastic layer that reflects a porous pattern of a dermatological area of interest on the human user.

The elastic layer, when cured, is tinted. To enable this, the silicone material includes a pigment dispersed within the silicone material. The pigment is designed to tint the resulting elastic layer according to a selected skin tone. That is, in some embodiments the silicone is pre-tinted prior to curing.

In one aspect of the kit, a flocking agent is provided. The flocking agent is pre-mixed into the silicone mixture. The flocking agent is designed to provide texture, such as a visual texture, so that the elastic layer visually blends in with the user's skin. The flocking agent may comprise natural hair, natural fibers, or synthetic fibers.

The silicone container may be ajar from which a user can scoop a needed amount of material. Alternatively, the silicone material may reside in a pump container. More preferably, the silicone container is a cartridge in a dispenser. The dispenser may be either a dispensing gun or a syringe. In either instance, the dispenser is used to extrude the silicone material onto the user's skin from the silicone mixture container.

The method further includes applying one of the at least one skin texturing pads to the applied silicone mixture. This is done before the silicone mixture cures. In this way, texture is created along the silicone material. Thereafter, the method comprises allowing the silicone material to cure on a skin surface of the human user. The silicone mixture becomes removably bonded to the user's skin as it cures. In some embodiments the texturing pad is contacted with the silicone on the skin for from 15 sec to 10 minutes, or from 30 sec to 5 minutes or from 1 minute to 3 minutes. Alternatively the texturing pad is contacted with the silicone on the skin for less than 10 minutes, less than 7 minutes, less than 5 minutes, less than 4 minutes, less than 3 minutes, less than 2 minutes or less than 1 minute. Alternatively, the texturing pad is contacted with the silicone on the skin for around 1, 2, 3, 4, 5, 6, 7, 8, 9 or 10 minutes.

Other kits may be used with the method. For example, in an alternate embodiment the second cosmetic kit comprises a first syringe having at least a single volume within a package. A viscous silicone mixture resides within the first volume. In some embodiments an adhesion promoter is included in the volume with the silicone mixture. In some embodiments a second volume contains a catalyst that is mixed with the silicone mixture. This silicone material is tinted to match a selected skin tone. Preferably, the silicone material is tinted by a pigment dispersed within the silicone mixture. The pigment may be a pigmented silicone oil, a pigmented cosmetic cream, or a pigmented powder.

The silicone material further has a flocking agent dispersed therein to provide visual texture. In this way the silicone material visually blends in to the user's skin upon curing.

The alternate cosmetic kit may further have at least one skin texturing pad. The skin texturing pad is fabricated with dimples for creating texture to the translucent elastic layer while the silicone material is curing. In this way, dimples are formed in the elastic layer that reflects a porous pattern of a dermatological area of interest on the human user.

The alternate cosmetic kit also includes a box or other packaging. The box may contain at least the syringe, and the skin texturing pad. A plurality of syringe tips may also be provided in the box. Each tip is configured to attach to a distal end of the syringe in order to facilitate dispensing of the silicone material.

The alternate cosmetic kit may further comprise at least a second syringe and optionally a third syringe or more. Each of the first, second, and third syringes has a volume that contains a silicone mixture. The silicone mixture in each syringe is tinted by a pigment, wherein the silicone material in each of the first, second and third syringes is tinted to match a different selected skin tone. In this way, a purchaser of the kit may readily choose a syringe that most closely matches his or her skin tone.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 depicts a syringe as described herein filling a blemish on the face of a user.

FIG. 2 depicts a texturizing pad applied to the silicone mixture in the blemish on the face of a user.

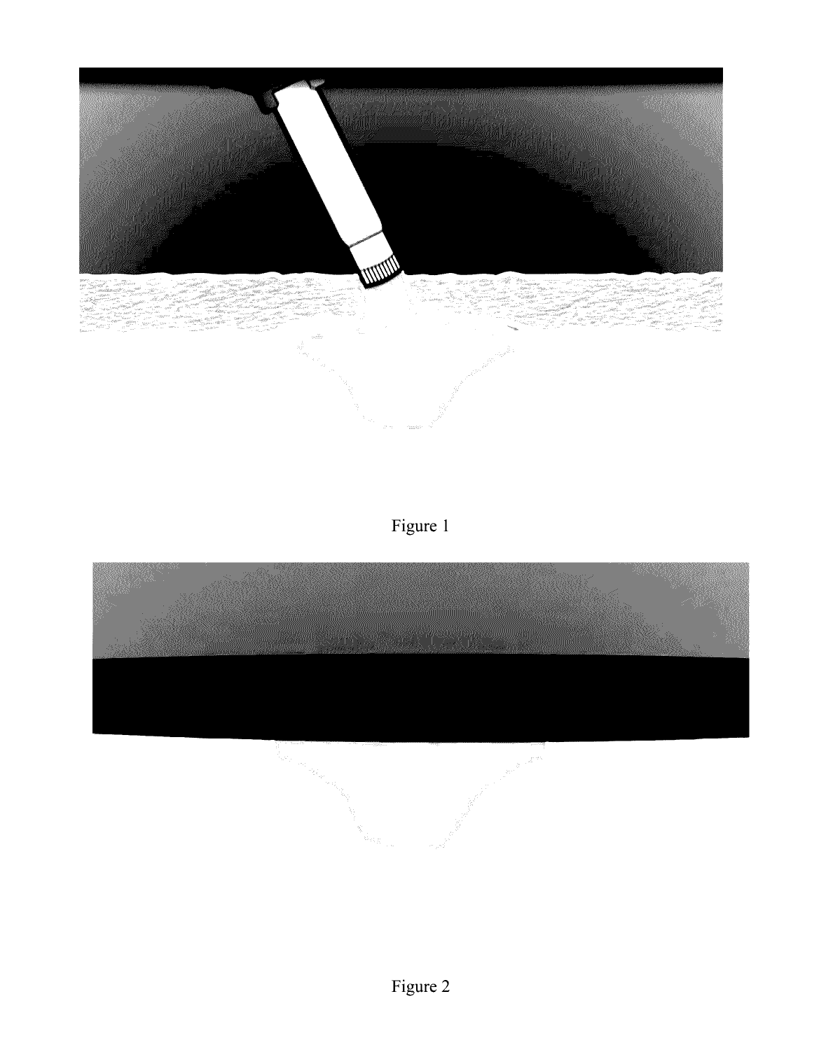

### DESCRIPTION OF CERTAIN EMBODIMENTS

### Definitions

As used herein, the term “flocking agent” refers to a collection of tiny spikes or hairs. The flocking agent may be fabricated from, for example, short natural or synthetic hair or fibers. The fibers are preferably colored.

The term “package” refers to any container that holds components for a cosmetic system. A package may be a small cardboard box or a box fabricated from other cellulosic material. Alternatively, the package may be fabricated from plastic or polycarbonate material.

The term “silicone material” refers to any viscous composition having a polymerized siloxane, or polysiloxane. Polysiloxanes have a silicon-oxygen backbone (Si—O—Si—O—Si—O . . . ) and an attached organic group (such as, but not limited to, methyl, ethyl or phenyl). The silicone material may be, for example, a linear silicone oil of the non-cross-linked polydimethylsiloxane (PDMS) type. Alternatively, the silicone material may be, for example, of the cross-linked organosiloxane type. The silicone material may be in the form of an emulsion. Suitable emulsions include but are not limited to water-in-silicone emulsions, oil-in-water emulsions, and silicone-in-water emulsions.

The term “adhesion promoter” refers to any monomeric silane moiety that acts as a coupling agent between a substrate and the silicone elastomer.

The term “catalyst” refers to any chemical moiety that refers to a material which, when combined with silicone, cures to form a substantially solid material. A non-limiting example is a platinum-type catalyst.

The term “dispenser” refers to any apparatus that dispenses a silicone material and a catalyst in such a manner that the silicone material and the catalyst are available for mixing and curing. A non-limiting example is a double-barrel syringe that allows the materials to be simultaneously extruded and mixed.

The term “silicone mixture” refers to a pre-mixed combination of silicone material, adhesion promoter, and catalyst.

The term “cured silicone” refers to a silicone that is cured in the presence of a catalyst, such as, but not limited to platinum. As such a platinum cured silicone is a silicone that is cured by platinum.

### DESCRIPTION OF SELECTED SPECIFIC EMBODIMENTS

In view of the deficiencies for covering dermatological blemishes and defects, there exists a significant need for alternative and improved methods to enable a user or patient to cover such blemishes and/or defects. An improvement in disguising skin defects was disclosed in the patent application U.S. 2012/0192886 A1, entitled “Methods for Disguising Dermatological Blemishes”. This application disclosed methods to make a silicone elastomer by application of a viscous silicone material and of a catalyst. Also disclosed are several types of dispensers and mixing tips to facilitate mixing of the silicone material and catalyst. The resulting silicone elastomer is a translucent, realistic artificial skin that effectively disguises dermatological defects such as scarring or acne, which defects cannot be effectively covered by traditional makeup. Furthermore, the methods described kits that can be purchased and used directly by a consumer in a convenient fashion.

Despite the major advances disclosed in patent application U.S. 2012/0192886 A1, areas for improvement still remained. A need exists for a single component silicone material which would be simpler and more convenient to apply and that would still provide excellent disguising of dermatological defects. Another need exists for a primer-less method that would not require application of a primer to the dermatological area of interest before applying the silicone material.

The present invention provides improved methods for disguising dermatological defects and blemishes. The methods employ a unique cosmetic system in the form of a kit that is provided or sold to a user in a box or package. The cosmetic system is a silicone-based system that can be used to temporarily fill acne or other dermatological blemishes. Alternatively, the cosmetic system may be used to disguise more significant scars that may arise from surgeries, diseases or traumatic accidents. The cosmetic system first comprises a silicone mixture, comprised of silicone material, adhesion promoter, and catalyst. In one embodiment the components may be packaged separately, for instance having the silicone material mixed with the adhesion promoter while the catalyst is packaged separately and mixed prior to application. Alternatively, all three components may be packaged separately. In one embodiment, the silicone material is fabricated from a dimethyl silicone such as polydimethylsiloxane. Such silicones are available, for example, from Applied Silicones, NuSil Technology, Momentive Silicones, Dow Corning, Wacker Silicones, Bluestar Silicones, Shin-Etsu, or AB Silicones. However, the cosmetic systems herein are not limited to any particular type or source of silicone material, so long as the silicone material may be catalyzed or cured at room temperature. In some embodiments, however, certain a user may find use in using a silicone that requires elevated heat in which case a heating element, such as a blow dryer, may be used to increase the temperature of the silicone mixture and speed curing. Preferably, the silicone material has the viscosity of a gel or soft solid at room temperature conditions, after catalysis.

Unexpected advantages of the compositions and methods disclosed here include the rapid application of a blemish concealer that does not require initial application of a primer. Ease of use and compatibility with existing makeup routines is also a benefit. Users of other blemish concealers identified an inconvenience in applying a primer that would run down their faces interfering with makeup routines. Eliminating the primer as disclosed herein eliminates this issue. An additional improvement is in safety and handling. Components of prior blemish concealers caused discomfort on the skin. This has been eliminated using the compositions of the present disclosure. Moreover, shipping is facilitated with the compositions of the present disclosure as the flammable liquid used in previously used blemish concealers has been removed. Accordingly, the present disclosure provides compositions and methods exhibiting unexpected improvements and benefits when compared to prior systems. For instance, principles of the present disclosure provide compositions and methods for concealing dermatological blemishes with improved comfort as compared to the discomfort of prior blemish concealers that required the use of a primer(s). In addition, principles of the present disclosure provide compositions and methods for shipping dermatological concealers including kits as described herein that are safer than shipping dermatological concealers that require the use of a primer(s).

In one embodiment the desired silicone material is combined with an oil, such as but not limited to a 50 centistoke oil. The oil serves as a diluent to facilitate extrusion through a dispenser. Stated another way, the diluent reduces the viscosity of the silicone material. At higher percentages, the diluent also softens the final cured product, making it more pliable. This is of benefit for larger patch applications on a user's skin.

In some embodiments the silicone provides high viscosity and temperature tolerance and good optical clarity upon mixing and curing.

In some embodiments the silicone offers a fast-curing, medical grade, silicone elastomer. The elastomer cures with a translucent appearance in just about 5 minutes, and offers a tensile strength that is generally about 550 psi with a tensile strength range of around 200 psi to around 1000 psi.

In another aspect of this first embodiment, the adhesion promoter is a monomeric silane moiety that acts as a coupling agent between the substrate and the silicone elastomer. However, the cosmetic systems herein are not limited to any particular type or adhesion promoter, so long as the adhesion promoter may act a coupling agent between substrate and silicone elastomer.

Several particularly desired adhesion promoters are tetrapropyl orthosilicate, 3-aminopropyl trimethoxysilane, 3-glycidoxypropyl trimethoxysilane, and vinyltrimethoxysilane. One preferred adhesion promoter is 3-glycidoxypropyl trimethoxysilane.

The adhesion promoter comprises between 0.1% and 10.0% of the mixture. In one embodiment, the adhesion promoter comprises between 0.5% and 5.0% of the composition.

In yet another aspect of this first embodiment, the catalyst is not limited to any particular type or source of catalyst. A preferred catalyst is a platinum metal catalyst but can also be a rhodium or palladium metal catalysts.

The catalyst comprises between 0.01% and 1.0% of the mixture. In some embodiments the catalyst comprises between 0.1% and 0.5% of the mixture. In some embodiments the catalyst comprises between 0.1% and 0.25% of the mixture.

In one aspect, the silicone mixture is custom-blended. This means that the mixture includes a cosmetic pigment that has been added to match a selected skin tone. Using pigments, various tints or shades of silicone mixture may be provided in different containers so that customers may select an intrinsically colored product that most closely matches their own skin tone.

As an alternative, the pigment may be a powder such as a cosmetic make-up powder. Alternatively, the pigment may be a silicone pigment. Various tones or colors of silicone pigment are available from NuSil Technology. Also, Eager Plastics, Inc. of Chicago, Ill. provides opaque silicone pigments having no/low odor and toxicity.

The pigment may be pre-mixed with the silicone material. Alternatively still, the pigment may be supplied as a separate component and mixed with both the silicone material. In this latter instance, the pigment will need a carrier material such as a low-viscosity silicone compound.

The cosmetic system optionally also includes a flocking agent. The flocking agent is used to provide depth and visual texture to the cosmetic mixture. The flocking agent may also provide color. Preferably, the flocking agent is pre-mixed with the silicone material. Alternatively still, the flocking agent may be supplied as a separate component with a pigment, and then mixed with the silicone material at the time the silicone material is applied by a user.

The flocking agent is preferably in the form of rayon fibers that are cut in very short sections, such as 5 to 25 mm. However, any material that is capable of providing texture to the silicone material may be used. Non-limiting examples include human hair, natural fibers, e.g., cotton and pulp, or synthetic fibers. The flocking agent may be of any color. However, in one aspect the flocking agent has a brown or black color so that flocking agent may be added to the silicone material to selectively darken the final mixture.

A very small amount of a thickening agent may be included with the silicone material. This is for the purpose of suspending the rayon fibers or other flocking agent in the viscous material. An example of a suitable thickening agent is the so-called Cabosil thickener. The term “cabosil” is an abbreviation for colloidal silica. Cabosil is a silica-based thickener that increases the viscosity of silicone. The silica-based thickener is desirable in the present application as it has been found that it will not change colors during the catalysis process. Cabosil is available from various sources, but is believed to be manufactured by Dow Chemical Company of Midland, Mich. and/or Cabot Corporation of Boston, Mass.

Another example of a thickening agent is a thixotropic agent. A thixotropic agent maintains a gel in a substantially solid or gelatinous phase during a sedentary state, but permits a reduction in viscosity when shaken or otherwise disturbed. The thixatropic agent also assists in suspending the rayon (or other) fibers. This prevents a condition known as “slumping.”

In order to dispense the silicone material onto a patient's skin, a dispenser is preferably employed. Thus, in one embodiment the cosmetic system includes a syringe for dispensing the silicone mixture. The syringe may be a double chambered or double barrel syringe in which one chamber contains the silicone and adhesion promoter, while the other contains the catalyst. Upon extrusion of the components of the syringe, the two components mix. The volume of the syringe terminates a tip, through which the viscous silicone mixture is extruded. The silicone material may be thus extruded directly onto a human user's skin.

The dispenser may alternatively be a dispensing gun. The dispensing gun also may be a double chambered applicator that receives one cartridge containing the silicone material and one cartridge containing the catalyst. In some embodiments the silicone material is mixed with an adhesion promoter in one of the chambers and mixed with the catalyst prior to application to the skin of the user.

It is understood that any dispensing apparatus capable of moving the viscous components of the cosmetic system from a dispenser may be employed.

The silicone mixture may be applied to the patient's skin in different ways. First, the mixture may be applied directly to the skin as the mixture is extruded from the tips of a syringe dispenser. Alternatively, the mixture may be squeezed onto a dish and then applied to the skin by use of a spatula or other tool. Alternatively still, the mixture may be squeezed into the user's hand or into a dish, and then applied to the area of concern by hand. In any instance, the silicone mixture takes about from 1 to 10 minutes to cure. In some embodiments it takes less than 5, 4, 3, 2 or 1 minute to cure. Upon curing, the mixture constitutes a solid synthetic elastomer. The resulting translucent elastic layer will stay on the patient's skin for several days, even if the user takes a shower or goes swimming.

The cosmetic system may also comprise a skin texturing pad. One embodiment of the skin texturing pad is a thin, flexible substrate having a plurality of dimples. In such a pad, the dimples are large and may be used to provide texture to a user's hand, arm, legs or foot, for example. Thus, the pad has a texture designed to match the porous pattern of the dermatological area of interest.

A separate skin texturing pad may be used on dermatological areas having finer features. Such a skin texturing pad again has a thin, flexible substrate having a plurality of dimples. However, in this version of a pad, the dimples are more subtle, and may be used to provide texture to a user's face, neck, or stomach, for example. In either instance, the use of a skin texturing pad tends to reduce the shine or light reflection from the cured silicone material.

The pads are preferably fabricated from polyurethane or thermoplastic elastomer. After the pad has been applied to the silicone, it is carefully removed. A matte skin cover disguising a dermatological blemish is left. The final material is dry to the touch, transparent, water resistant, durable, flexible, and has a natural texture. Under normal conditions it is anticipated that the dried material will last up to from around 12 to around 72 hours.

It is preferred that the components of the cosmetic system are provided or sold as a kit in a single package. The packaging is referred to herein as a box.

It is understood that the term “box” includes any form of suitably-sized packaging. A box is not limited to a traditional cardboard or plastic box having a lid, although such is preferred; instead, the term “box” may also include a blister pack or a bag.

In any aspect, the box holds the various components making up the cosmetic system. These may include:
a dispenser (such as a syringe);
at least one volume or cartridge containing the silicone mixture;
a plurality of removable tips;
one or more skin texturing pads.

One non-limiting example of a box is a collection of components that represent a cosmetic system. The collection of components begins with a set of two or three syringes, with each containing a pre-loaded volume of tinted silicone mixture. The first volume of silicone mixture is tinted to match a first selected skin tone; the second volume of silicone mixture is tinted to match a second selected skin tone; and the third volume of silicone mixture is tinted to match a third selected skin tone. In this way, the user may experiment with the different syringes to determine which one most closely approximates their own skin tone. More than 3 syringes and tones also may find use in the embodiments described herein. Preferably, the box will include two, three or even more sets of cartridges containing the viscous silicone mixture.

The box may also contain a spatula, which has a tip and may be used to applying the silicone mixture onto the user's skin. Preferably, a cosmetic kit will have two or more spatulas.

Based, on the above-described cosmetic system and components, a method is provided herein for disguising dermatological blemishes on a human user. In one aspect, the method includes providing a package containing a cosmetic system. Generally, the system will include a viscous silicone mixture. The silicone mixture is preferably tinted with a pigment according to a desired skin tone. In one aspect, the cosmetic system also includes a flocking agent. The flocking agent is pre-mixed with the silicone mixture to provide visible texture.

The package may also have at least one skin texturing pad. The skin texturing pads are textured in accordance with the dermatological area of interest. Ideally, the pads are created from molds that, in turn, have been created from impressions taken on actual physical skin.

In one aspect, the method also includes applying one of the at least one skin texturing pads to the applied silicone mixture before the silicone mixture cures. This creates texture to the silicone mixture. The method may include selecting a particular texturing pad having dimples to match a skin pattern of the dermatological area of interest.

Using the texture pads, a user is able to impart texture to the silicone mixture to the mixture before it cures. In this way, the mixture creates a seamless blend between the defect site and the surrounding flesh as it cures. Even the non-artist can obtain a nice blend into the skin with little to no training. The skin texturing pad is then removed from the user's skin before the silicone mixture fully cures. As such, in some embodiments the skin texturing pad is contacted with the silicone on the skin of a user within about 30 seconds of application to the skin. The pad imparts texture to the silicone before it has fully cured, then after the silicone has substantially cured and bonded with the skin, the pad can be removed. In some embodiments the texturing pad is contacted with the silicone on the skin for from 15 sec to 10 minutes, or from 30 sec to 5 minutes or from 1 minute to 3 minutes. Alternatively the texturing pad is contacted with the silicone on the skin for less than 10 minutes, less than 7 minutes, less than 5 minutes, less than 4 minutes, less than 3 minutes, less than 2 minutes or less than 1 minute. Alternatively, the texturing pad is contacted with the silicone on the skin for around 1, 2, 3, 4, 5, 6, 7, 8, 9 or 10 minutes.

The method includes allowing the silicone mixture to cure on a skin surface of the human user. The silicone mixture becomes bonded to the user's skin.

In this method, it is preferred that the silicone mixture is provided in the volume of a syringe. Preferably, the volume is sized such that the silicone mixture is dispensed at a reasonable speed. In one aspect, the user selects from a first syringe, a second syringe, and a third syringe in a package, wherein each of the first, second, and third syringes has a silicone mixture that is tinted according to a selected skin tone. In this way, the user may create a cured, translucent covering that is tinted most closely according to their skin.

In one aspect, the user chooses a set of syringes from two or three or more different sets of syringes. For example, a first set of syringes may be tinted for Caucasian skin tones, a second set of syringes may be tinted for ethnic skin tones, and a third set of syringes may be tinted for Asian skin tones, although the tints need not be related to any ethnic group. Rather, the tints reflect a range of skin tones. Each of the two or three sets, in turn, has two or three or more syringes offering light, medium and dark tints. The patient selects the “kit” that most closely matches his or her skin tone.

The method may also include applying facial makeup to the dermatological area of interest prior to applying the silicone mixture to the dermatological area of interest. It is noted that the silicone mixture dries translucently, and additional makeup need not be placed over the silicone material. However, it is also within the scope of the present disclosure to provide a mineral powder based make up or liquid make up that man be applied to the cured silicone layer. The custom blend of the silicone mixture and the pigment is preferably designed so that an element of translucence is maintained. Ideally, the cured silicone mixture should not interfere with the user's skin appearance, but should blend in so as not to be noticed. However, in some instances a user may have a scar that has left a different skin coloration than their natural skin. In this instance, the user may choose to use a special makeup that blends in the color of the scar tissue with the surrounding skin.

Ancillary to the cosmetic system is a consideration as to pigmentation for users in the market place. The human race presents a true “kaleidoscope” of colors. In one aspect, it is desirable to select a minimum number of pre-mixed syringes which nevertheless support the “skin kaleidoscope” presented by the marketplace. In another aspect, it is desirable to offer a color-matching system wherein a silicone material is custom blended specifically for an individual user by using a computer-driven color matching system.

The foregoing description and examples have been set forth merely to illustrate the inventions herein and are not intended to be limiting. Since modifications of the disclosed embodiments incorporating the spirit and substance of the inventions may occur to persons skilled in the art after reading this disclosure, the inventions should be construed broadly to include all variations falling within the scope of the appended claims and equivalents thereof.

### EXAMPLES

Examples of the silicone mixture are mixtures of silicone elastomer, adhesion promoter, and vinylsiloxane complexed platinum catalyst. Several adhesion promoters were evaluated. Adhesion promoters tested included tetrapropyl orthosilicate, 3-aminopropyl trimethoxysilane, 3-glycidoxypropyl trimethoxysilane, and vinyltrimethoxysilane. In order to evaluate the silicone mixtures' efficacies, a qualitative test was implemented where the time required for the edges of the applied silicone patch would take to lift from the skin after curing at room temperature. In addition, once the edges of the silicone had lifted, the patch was manually peeled away to qualitatively determine the amount of adhesion.

The testing procedure was as follows. Equal parts of silicone elastomer were thoroughly mixed with platinum catalyst and adhesion promoter and a thin layer of silicone, approximately one inch in diameter, was spread onto a subject's forearm and allowed to cure at room temperature. The above referenced adhesion promoters were mixed into the silicone formulation at 1.0% to 1.5% concentration and a catalyst. Each of the adhesion promoters was evaluated individually and the results are described in the following table.

| | | Adhesion | | | |
|---|---|---|---|---|---|
| | | Promoter | | | Adhesion to |
| Silicone Elastomer | Adhesion Promoter | Concentration | Edge Lifting | | Skin |
| Silicone Elastomer | None | 0.0% | 1 | hour | poor |
| Silicone Elastomer | tetrapropyl | 1.0% | 5 | hours | good |
| | orthosilicate | | | | |
| Silicone Elastomer | tetrapropyl | 1.5% | 5 | hours | good |
| | orthosilicate | | | | |
| Silicone Elastomer | 3-aminopropyl | 1.0% | 3.25 | hours | good |
| | trimethylsiloxane | | | | |
| Silicone Elastomer | 3-aminopropyl | 1.5% | 3.25 | hours | good |
| | trimethylsiloxane | | | | |
| Silicone Elastomer | 3-glycidoxypropyl | 1.0% | >24 | hours | very good |
| | trimethoxysilane | | | | |
| Silicone Elastomer | 3-glycidoxypropyl | 1.5% | >24 | hours | very good |
| | trimethoxysilane | | | | |
| Silicone Elastomer | vinyltrimethoxysilane | 1.0% | 2 | hours | good |
| Silicone Elastomer | vinyltrimethoxysilane | 1.5% | 2.5 | hours | good |

1. A composition comprising a silicone material, an adhesion promoter, wherein said adhesion promoter is a monomeric silane moiety at a concentration of from about 0.5% to 5% (w/w), and a catalyst, for use in disguising dermatological blemishes.

2. The composition of claim 1, wherein the silicone material is a cured silicone elastomer.

3. The composition of claim 2, wherein the cured silicone elastomer is a platinum cured silicone elastomer.

4. (canceled)

5. (canceled)

6. The composition of claim 1, wherein the monomeric silane moiety comprises 1.0% to 1.5% (w/w) of the composition.

7. The composition of claim 1, wherein the monomeric silane moiety is selected from the group consisting of tetrapropyl orthosilicate, 3-aminopropyl trimethoxysilane, 3-glycidoxypropyl trimethoxysilane, and vinyltrimethoxysilane.

8. The composition of claim 7 wherein the monomeric silane moiety is 3-glycidoxypropyl trimethoxysilane.

9. The composition of claim 1, wherein the catalyst is a transition metal catalyst for the polymerization of the silicone material into an elastomeric patch.

10. The composition of claim 9, wherein the transition metal catalyst comprises 0.01% to 1.00% (w/w) of the composition.

11. The composition of claim 10, wherein the transition metal catalyst comprises 0.1% to 0.5% (w/w) of the composition.

12. The composition of claim 9, wherein the catalyst is a transition metal catalyst complexed with vinylsiloxane.

13. The composition of claim 12, wherein the transition metal catalyst is a platinum catalyst complexed with vinylsiloxane.

14. The composition of claim 1, further comprising a flocking agent.

15. The composition of claim 14, further comprising a thixotropic agent or a thickener or both.

16. A method for disguising dermatological blemishes on a human user, comprising
a. opening a package containing at least:
i. a container for the silicone mixture material;
ii. a viscous silicone mixture composition of claim 1 within the silicone container;
iii. at least one skin texturing pad, being designed with dimples for creating texture to translucent elastic layer such that the dimples generally reflect a porous pattern of dermatological interest on the human user;
iv. wherein:
1. the silicone mixture material is tinted by a pigment dispersed with the material;
2. and the pigment is designed to tint the elastic layer according to a selected skin tone;
b. applying the silicone mixture material on the dermatological area of interest;
c. applying at least one of the texturing pads to the silicone material applied to the dermatological area of interest, in order to create texture to the silicone material; and
d. allowing the silicone mixture material to cure on a skin surface of a human user, wherein the silicone material becomes bonded to the human user's skin.

17. The method of claim 16, wherein the container for the silicone mixture material is a cartridge configured to be loaded into a dispensing gun;

18. The method of claim 16, wherein the silicone mixture material is tinted according to the selected skin tone;

19-35. (canceled)

36. A kit for disguising dermatological blemishes on a human user, comprising at least a first and second silicone mixture each being a different tint, wherein each silicone mixture comprises a silicone material, an adhesion promoter, wherein said adhesion promoter is a monomeric silane moiety at a concentration of from about 0.1% to 10% (w/w), and a catalyst.

37. A method for disguising dermatological blemishes on a human user, comprising:
a. applying a composition according to claim 1 to the dermatological area of interest of a user in need thereof, wherein no primer is added to the dermatological area of interest;
b. applying a texturing pad to the silicone material applied to the dermatological area of interest, in order to create texture to the silicone material; and
c. allowing the silicone mixture material to cure on a skin surface of a human user, wherein the silicone material becomes bonded to the human user's skin.
